(19) United States
(12) Patent Application Publication
(10) Pub. No.: US 2019/0365634 A1
(43) Pub. Date: Dec. 5, 2019

(54) Human Axilla Cleansing Compound

(71) Applicant: Boyle's Naturals, LLC, Weatogue, CT (US)

(72) Inventor: Katie E. Boyle, Weatogue, CT (US)

(73) Assignee: Boyle's Naturals, LLC, Weatogue, CT (US)

(21) Appl. No.: 15/990,893

(22) Filed: May 29, 2018

(51) Int. Cl. A61K 8/92; A61K 8/73; A61K 8/67; A61K 8/19; A61Q 19/10

(52) U.S. Cl. CPC A61K 8/922; A61K 8/732; A61K 8/678; A61K 8/19; A61K 2800/74; A61K 2800/28; A61K 2800/522; A61K 2800/524; A61Q 19/10

(57) ABSTRACT

A compound for the cleansing of human underarm skin includes a natural cleansing agent, a natural antibacterial agent, a natural abrasive agent, a natural bulking agent, a natural preservative, a natural thinning agent, a natural antioxidant agent, a natural anti-mold agent, and a natural anti-viral agent.

### FIELD OF THE INVENTION

The invention relates to skin cleansing compounds. More specifically, the invention is related to skin cleansing and exfoliating compounds for use in the human axilla, especially employing only natural (non-man made) ingredients in effective combinations.

### BACKGROUND

With the lack of regulations controlling ingredients permitted for use in consumer personal care products and cosmetics in the United States, consumers are regularly (and most of the time unknowingly) exposed to ingredients that may be linked to disease. Current consumer options for underarm body odor control include:
a. Simply washing with soap and water;
b. Using an over the counter or prescription-level deodorant/anti-perspirants (hereafter denoted as “CD/APs”) that contain harsh sweat-blocking and odor-killing ingredients such as aluminum chlorohydrate, aluminum chlorohydroxide, triclosan, paraben preservatives, synthetic fragrances and phthalates, studied for their potential link to human endocrine system disruption and cancers, specifically breast cancers;
c. Employing more invasive underarm dermatological procedures such as Botox or surgical removal of sweat glands in a medical office or hospital;
d. Applying one of numerous natural underarm deodorants (hereafter denoted as NDs) introduced to the marketplace in recent years that are inherently less effective at reducing underarm body odor due to the intentional omission of the harsh sweat-blocking and odor-killing ingredients mentioned in bullet b. above.

It is the present inventor's belief that washing with soap and water is insufficient at fully and completely cleansing away bacteria and build-up from previous applications of deodorant, and there exists a need for a body scrub compound that cleanses better that simply washing with soap and water, and such is an objective of the invention. Cleaner underarms at the time of application of either CD/AP or ND will result in the product better controlling body odor.

It is the present inventor's belief, based on both peer reviewed studies and employing the precautionary principle in public health, that ingredients in CD/APs are in fact harmful to human health and should be avoided. Humans are exposed to toxins from products we apply to our bodies at all stages of life: as a developing fetus, an infant, child, adolescent and adult. The health risks of chemicals found in CD/APs may include increased rates of cancer, especially breast cancer, endocrine system disruption, abnormalities in reproductive function and anatomy, and neurodevelopmental delays in children. There exists a need to provide an underarm body scrub compound that aids in controlling body odor and that is nonharmful to human biology, and such is an objective of the invention.

It is the present inventor's belief that CD/AP's ingredients present health risks to not only the end user, but also to workers who come into contact with them during making and processing of the ingredient. For the protection of workers, there exists a need to reduce the consumer dependence on—and industrial production of—these harsh chemicals, and such is an objective of this invention.

It is the present inventor's belief that CD/AP's ingredients present health risks to not only the end user and workers who come into contact with them, but to the environment in and around the plant where they are processed and all along the supply/transport chain. For the protection of our environment, there exists a need to reduce the consumer dependence on—and industrial production of—these harsh chemicals, and such is an objective of this invention.

There exists a need for body odor control solutions for people seeking dermatological care that avoid invasive procedures, which carry them risk of infection, injury or worse.

It is the present inventor's belief that NDs, while heathier for the human body than CD/APs, are less effective and therefore do not adequately control body odor to the extent most consumers desire. There exists a need to provide an all-natural underarm body scrub compound which is effective at cleansing the underarm, and thereby assists NDs to meet consumer demand for body odor control over the course of the day, and such is an objective of the invention.

There exists a need to provide an all-natural underarm body scrub compound which is pleasurable to use, and such is an objective of the invention.

There exists a need to provide an all-natural underarm body scrub compound which is economical to use, and such is an objective of the invention.

There exists a need to provide an all-natural underarm body scrub compound which is non-polluting, and such is an objective of the invention.

There is a gap in available natural body odor control products in the marketplace for the following types of consumers in particular:
Consumers who struggle with the social stigma of body odor,
Natural-minded consumers unsatisfied by the effectiveness of NDs,
Consumers who exercise and sweat,
Pre-pubertal teens who have begun to have body odor but aren't ready to be buying and applying CD/AP every day,
New moms with little time for self-care,
Breastfeeding moms concerned with the transfer of toxins into their breastmilk,
Those with a new breast cancer diagnosis who have been asked by their doctors to stop using CD/AP,
Dermatologists who have patients considering prescription level CD/APs or other very invasive procedures.

Additional needs and objectives will become apparent by a review of the following disclosure.

### SUMMARY OF THE INVENTION

The invention may be embodied in, or practiced using, an underarm skin cleansing compound which is all-natural; more effective than just washing with soap; not harmful to the health of the end user, plant worker or the environment; not requiring a prescription; that permits user to delay or avoid invasive medical procedures; that use of which prior to applying ND is more effective than using ND alone; that is pleasurable and economical to use; and may be made of one or more of such ingredients as castile soap (distilled water, saponified oils of olive, coconut and castor oils, and vegetable glycerin), cinnamon, aluminum-free baking soda, corn starch, essential oils, vitamin E, rosemary oleoresin, and distilled water.

The invention may be embodied in, or practiced using, an underarm skin cleansing compound including a natural cleansing agent, a natural antibacterial agent, a natural abrasive agent, a natural bulking agent, a natural preservative, a natural thinning agent, a natural antioxidant agent, a natural anti-mold agent, and a natural anti-viral agent.

It should be understood that the foregoing summary, as well as the following detailed description, are merely exemplary and the invention is not limited to the precise arrangements and instrumentalities shown.

### DESCRIPTION OF EXEMPLARY EMBODIMENTS

Referring to the following list of ingredients for a preferred exemplary embodiment, a compound for underarm skin cleansing is provided which is all-natural; more effective than just washing with soap; not harmful to the health of the end user, plant worker or the environment; not requiring a prescription; that permits user to delay or avoid invasive medical procedures; that use of which prior to applying ND is more effective than using ND alone; and that is pleasurable and economical to use.

| | | | | | Less |
|---|---|---|---|---|---|
| | | | Wet/ | | Preferable |
| Ingredient: | Primary Function | Ancillary Functions | dry | % | Range |
| Distilled | Thinning | | W | 21 | 15-22% |
| Water | | | | | |
| Fragrance- | Cleansing | Adds oils to paste which makes | W | 11 | 10-12% |
| free Castile | | it have more of a lather when | | | |
| Soap* | | rubbing on underarm. | | | |
| Cinnamon | Antibacterial | Dries underarm skin (removing | D | 1.4 | 1-1.5% |
| | | sweat) | | | |
| Aluminum- | Abrasive - for | Cleansing | D | 43.8 | 43-60% |
| free Baking | skin/Exfoliating the | | | | |
| Soda | underarm skin to | | | | |
| | remove bacteria and | | | | |
| | build up | | | | |
| Corn Starch | Bulking - (less | | D | 21 | 20-25% |
| | expensive than | | | | |
| | baking soda.) | | | | |
| Organic | Antibacterial | Also adds a pleasing, natural | W | 0.03 | .03-.06 |
| Geranium | | (non-synthetic) fragrance | | | |
| Essential Oil | | | | | |
| Organic Tea | Antibacterial | Also adds a pleasing, natural | W | 0.03 | .03-.06 |
| Tree | | (non-synthetic) fragrance | | | |
| Essential Oil | | | | | |
| Vitamin E | Natural preservative: | | W | 1 | .9-1.1% |
| T-50 Oil | antioxidant, | | | | |
| (Tocopherol)** | Antibacterial; anti | | | | |
| | mold; anti-viral | | | | |
| Rosemary | Natural preservative: | | W | .05 | .045-.065% |
| Oil Extract | Antioxidant; anti | | | | |
| (ROE) | mold; anti-viral | | | | |
| (rosemary | | | | | |
| oleoresin)*** | | | | | |

*Castile soap ingredients: distilled water, saponified oils of olive, coconut and castor, vegetable glycerin
**Vitamin e oil is tocopherol T-50 and also contains soybean oil
***Rosemary oil extract contains: Carnosic Acid, Rosmarinic Acid, Rosmaridiphenol, Carnosol, Rosmanol, and Rosmaridiquinone.

Use of the above compound provides at least the following benefits;
Naturally lathers and cleanses away odor causing bacteria and build-up of previous day's applied deodorant, which results in a cleaner surface to apply deodorant and thus better body odor control.
Gently exfoliates away bacteria and build-up of previous day's applied deodorant which results in a cleaner surface to apply deodorant and thus better body odor control.
Uses the power of naturally anti-septic and anti-bacterial ingredients from the environment such as cinnamon, tea tree and geranium to kill body odor-causing bacteria.
Allows underarms to sweat, a normal body function, but controls odor-causing bacteria. The aluminum ingredients in CD/APs clog the user's pores, stopping them from sweating. Sweating is a normal body function that should be allowed to happen.
Enables consumers to successfully use ND (i.e. with perceived adequate body odor control).
Provides a few more hours of body odor protection to consumers who previously dealt with body odor soon after application of ND.
Allows people struggling with the social stigma of body odor another add-on option to control it.
Allows consumers to use the most readily available and affordable ND and removes the burden of “shopping around” for the perfect ND that works for them.
Allows men and women who exercise or consider themselves “athletes” another tool for reducing body odor during and after working out. It also allows consumers to exercise before social or professional situations and still maintain decorum, without body odor.
When added to teens' underarm skin cleansing routine, the compound is often sufficient alone to delay parents regular purchasing of ND or CD/AP at the point in which teens are approaching puberty and beginning to experience body odor.
Offers new mothers a solution for body odor control during the weeks/months in which the focus is often only on the new baby rather than on her own self-care. New mothers often also become more aware of toxins in their environment during these sensitives
Allows people with a recent breast cancer diagnosis, who are told by their doctor to stop using certain conventional, non-natural products such as CD/AP, to have an option for good body odor control.
Allows consumers seeking prescription level anti-perspirant another option to try before resorting to such strong chemical interventions;
Allows consumers seeking clinical, dermatologist-level care another option to try prior to resorting to the more invasive procedures such as Botox or surgical removal of sweat glands in the underarm.

Referring next to the following list of ingredients for an exemplary embodiment that is less-preferred than the first embodiment, but still within the invention, a compound for underarm skin cleansing is provided which is one or more of all-natural; more effective than just washing with soap; not harmful to the health of the end user, plant worker or the environment; not requiring a prescription; that permits user to delay or avoid invasive medical procedures; that use of which prior to applying ND is more effective than using ND alone; and that is pleasurable and economical to use.

| Ingredient: | Possible substitutes | Notes |
|---|---|---|
| Distilled Water | Could decrease amount or | If omitted water, vitamin E oil and ROE may be |
| | omit. | omitted because water is the thing that creates the |
| | | opportunity for bacteria to grow. |
| Fragrance-Free | Another similar castile | |
| Castile Soap | soap in same proportion. | |
| Organic Ground | Non-organic ground | |
| Cinnamon | cinnamon. | |
| Aluminum-free | Increase amount, in which | Other potential powders for exfoliating properties: |
| Baking Soda | case corn starch could also | juglans reglia (walnut shell) flour or arrowroot |
| | be omitted. | powder. Another potential powder for drawing out |
| | Another ground powder | impurities (detoxing) from skin is activated |
| | for some of the baking | charcoal powder. |
| | soda (see notes). | |
| Corn Starch | Could omit, in which case | |
| | more baking soda or one | |
| | of the powders noted as | |
| | substitutes for baking soda | |
| | would be used. | |
| Organic | Other antibacterial oils | |
| Geranium | (see notes) could be used | |
| Essential Oil | including eucalyptus, | |
| and/or | lavender, clove, patchouli, | |
| Organic Tea Tree | basil, rosemary, bergamot. | |
| Essential Oil | | |
| Vitamin E T-50 | Another natural | |
| Oil (tocopherol) | preservative | |
| Rosemary Oil | Another natural | |
| Extract (ROE) | preservative. | |
| (Rosemary | | |
| Oleoresin) | | |

Use of the above compound provides at least the following benefits;
Naturally lathers and cleanses away odor causing bacteria and build-up of previous day's applied deodorant, which results in a cleaner surface to apply deodorant and thus better body odor control.
Gently exfoliates away bacteria and build-up of previous day's applied deodorant which results in a cleaner surface to apply deodorant and thus better body odor control.
Uses the power of naturally anti-septic and anti-bacterial ingredients from the environment such as cinnamon, tea tree and geranium to kill body odor-causing bacteria.
Allows underarms to sweat, a normal body function, but controls odor-causing bacteria. The aluminum ingredients in CD/APs clog the user's pores, stopping them from sweating. Sweating is a normal body function that should be allowed to happen.
Enables consumers to successfully use ND (i.e. with perceived adequate body odor control).
Provides a few more hours of body odor protection to consumers who previously dealt with body odor soon after application of ND.
Allows people struggling with the social stigma of body odor another add-on option to control it.
Allows consumers to use the most readily available and affordable ND and removes the burden of “shopping around” for the perfect ND that works for them.
Allows men and women who exercise or consider themselves “athletes” another tool for reducing body odor during and after working out. It also allows consumers to exercise before social or professional situations and still maintain decorum, without body odor.
When added to teens' underarm skin cleansing routine, the compound is often sufficient alone to delay parents regular purchasing of ND or CD/AP at the point in which teens are approaching puberty and beginning to experience body odor.
Offers new mothers a solution for body odor control during the weeks/months in which the focus is often only on the new baby rather than on her own self-care. New mothers often also become more aware of toxins in their environment during these sensitives
Allows people with a recent breast cancer diagnosis, who are told by their doctor to stop using certain conventional, non-natural products such as CD/AP, to have an option for good body odor control.
Allows consumers seeking prescription level anti-perspirant another option to try before resorting to such strong chemical interventions;
Allows consumers seeking clinical, dermatologist-level care another option to try prior to resorting to the more invasive procedures such as Botox or surgical removal of sweat glands in the underarm.

The compound is an all-natural and gentle wash-off exfoliant for the underarm. It may be used in the shower or on-the-go, prior to applying deodorant, to better cleanse, remove build-up and inhibit odor-causing bacteria. Using the compound prior to applying deodorant maximizes the effectiveness of the deodorant, whether it be a ND or CD/AP.

The compound is a useful to anyone, but especially:
Consumers who struggle with the social stigma of body odor,
Natural-minded consumers unsatisfied by the effectiveness of NDs,
Consumers who exercise and sweat,
Pre-pubertal teens who have begun to have body odor but aren't ready to be buying and applying CD/AP every day,
New moms with little time for self-care,
Breastfeeding moms concerned with the transfer of toxins into their breastmilk,
Those with a new breast cancer diagnosis who have been asked by their doctors to stop using CD/AP,
Dermatologists who have patients considering prescription level CD/APs or other very invasive procedures.

Instructions for applying are: 1) dispense a dime-sized amount onto wet hand, 2) rub for 5-10 seconds into underarm, 3) rinse or wipe away, and 4) optionally apply deodorant as normal.

While the foregoing examples demonstrates just two of many possible examples for making or practicing the invention, such is not meant to be limiting, and the scope of the invention should only be judged according the following claims thereto, including all equivalent interpretation to which they are entitled.

I claim:

1. A compound for the cleansing of skin in human axilla comprising:
a natural cleansing agent;
a natural antibacterial agent;
a natural abrasive agent;
a natural bulking agent; and
a natural preservative.

2. The compound of claim 1 further comprising;
a natural thinning agent;
a natural antioxidant agent;
a natural anti-mold agent; and
a natural anti-viral agent.

3. The compound of claim 2 where
the natural cleansing agent is Castile soap;
the natural antibacterial agent is one or more of cinnamon, geranium essential oil, tea essential oil, tocopherol, and rosemary oleoresin;
the natural abrasive agent is aluminum-free baking soda;
the natural bulking agent is corn starch;
the natural preservative is tocopherol;
the natural thinning agent is distilled water;
the natural antioxidant agent is one or both of tocopherol and rosemary oleoresin;
the natural anti-mold agent is one or both of tocopherol and rosemary oleoresin; and
the natural anti-viral agent is one or both of tocopherol and rosemary oleoresin.

4. The compound of claim 3 wherein
the distilled water comprises 15-22% of the compound by volume;
the Castile soap comprises 10-12% of the compound by volume;
the cinnamon comprises 1-1.5% of the compound by volume;
the aluminum-free baking soda comprises 43-60% of the compound by volume;
the corn starch comprises 20-25% of the compound by volume;
the geranium essential oil comprises 0.03-0.06% of the compound by volume;
the tea tree essential oil comprises 0.03-0.06% of the compound by volume;
the tocopherol comprises 0.9-1.1% of the compound by volume; and
the rosemary oleoresin comprises 0.045-0.065% of the compound by volume.

5. The compound of claim 3 wherein
the distilled water comprises 21% of the compound by volume;
the Castile soap comprises 11% of the compound by volume;
the cinnamon comprises 1.4% of the compound by volume;
the aluminum-free baking soda comprises 43.8% of the compound by volume;
the corn starch comprises 21% of the compound by volume;
the geranium essential oil comprises 0.03% of the compound by volume;
the tea tree essential oil comprises 0.03% of the compound by volume;
the tocopherol comprises 1% of the compound by volume; and
the rosemary oleoresin comprises 0.05% of the compound by volume.

5. The compound of claim 2 where
the natural cleansing agent is a natural castile soap;
the natural antibacterial agent is one or more of cinnamon, geranium essential oil, tea essential oil, tocopherol, rosemary oleoresin, eucalyptus, lavender, clove, patchouli, basil, rosemary, or bergamot;
the natural abrasive agent is one or more of aluminum-free baking soda, juglans flour, arrowroot powder, and activated charcoal powder;
the natural bulking agent is one or both of corn starch and baking soda;
the natural preservative is tocopherol;
the natural thinning agent is distilled water;
the natural antioxidant agent is one or both of tocopherol and rosemary oleoresin;
the natural anti-mold agent is one or both of tocopherol and rosemary oleoresin; and
the natural anti-viral agent is one or both of tocopherol and rosemary oleoresin.
